(19) United States
(12) Patent Application Publication
(10) Pub. No.: US 2016/0374610 A1
(43) Pub. Date: Dec. 29, 2016

(54) HUNGER MANAGEMENT

(71) Applicant: KONINKLIJKE PHILIPS N.V., EINDHOVEN (NL)

(72) Inventors: BORIS EMMANUEL RACHMUND DE RUYTER, PEER (BE); BART KROON, EINDHOVEN (NL)

(21) Appl. No.: 15/187,816

(22) Filed: Jun. 21, 2016

Related U.S. Application Data
(60) Provisional application No. 62/183,247, filed on Jun. 23, 2015.

(51) Int. Cl. A61B 5/00; A61B 5/145; G01G 19/44; G09B 19/00; G09B 5/12; A61B 5/11; A61B 5/0205

(52) U.S. Cl. CPC A61B 5/486; A61B 5/4866; A61B 5/1118; A61B 5/14532; A61B 5/14546; A61B 5/0205; A61B 5/0022; A61B 5/6898; A61B 5/1112; G09B 19/0092; G09B 5/125; G01G 19/44; A61B 5/02405

(57) ABSTRACT

Methods and apparatus for providing individuals with coaching concerning their eating choices. The coaching system makes a determination of an individual's hunger, a determination concerning the availability of food for consumption and, in some embodiments, a determination of the individual's mood. Based on these determinations, the system makes eating recommendations to the individual, e.g., such as exhortations to refrain from eating or to eat a healthy snack.

### CROSS-REFERENCE TO RELATED APPLICATIONS

This patent application claims the priority benefit under 35 U.S.C. §119(e) of U.S. Provisional Application No. 62/183,247, filed on Jun. 23, 2015, the contents of which are herein incorporated by reference.

### TECHNICAL FIELD

The present invention relates to methods and apparatus for weight management, and in particular to methods and apparatus for coaching user behavior based on a measurement of the user's hunger state.

### BACKGROUND

Obesity is a widespread problem. According to the World Health Organization, worldwide obesity has nearly doubled since 1980. Thirty-five percent of adults aged 20 and over were overweight in 2008, and 11% were obese. Sixty-five percent of the world's population live in countries where obesity kills more people than malnutrition. More than 40 million children under the age of five were overweight in 2011.

The tragedy is that obesity is preventable. Several of the factors that contribute to obesity (poor eating choices, failure to exercise, etc.) result from poor choices. These contributing factors can be addressed by providing individuals with coaching that helps them make better choices. For example, many people pay personal trainers to encourage them to follow a regular regimen of exercise. Other people subscribe to programs that provide them with premade meals with nutritional content that is selected to facilitate weight loss.

Accordingly, there is a need for methods and apparatus that provide individuals with coaching concerning their eating choices.

### SUMMARY

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

Generally speaking, embodiments of the present invention relate to methods and apparatus for providing individuals with coaching concerning their eating choices. The coaching system makes a determination of an individual's hunger, a determination concerning the availability of food for consumption and, in some embodiments, a determination of the individual's mood. Based on these determinations, the system makes eating recommendations to the individual, e.g., such as exhortations to refrain from eating or to eat a healthy snack.

In one aspect, embodiments of the present invention relate to a method for hunger management. The method includes measuring a factor related to a user's metabolic state using a first sensor configured to provide a first measurement signal; measuring a factor related to the availability of food for consumption by the user using a second sensor configured to provide a second measurement signal; receiving the first measurement signal and the second measurement signal at a processing unit configured to make a determination of the user's hunger utilizing the received first measurement signal and the received second measurement signal; and providing feedback to the user concerning edible consumption tailored to the determination of the user's hunger. In one embodiment, the feedback is selected from the group consisting of a suggestion to eat a snack, a suggestion to refrain from eating, a message concerning the availability of unhealthy food, and an interactive distraction.

In one embodiment, the first sensor is selected from the group consisting of a ketone sensor, a glucometer, a semi-continuous weight sensor, and an activity sensor. In one embodiment, the second sensor is selected from the group consisting of a location sensor, an artificial nose, a social data analyzer, and a daily routine analyzer.

In one embodiment, the method further includes measuring a factor related to the user's mood using a third sensor configured to provide a third measurement signal and receiving the third measurement signal at the processing unit, wherein the processing unit is further configured to incorporate the third measurement signal into the determination of the user's hunger. The third sensor may be selected from the group consisting of a sensor measuring galvanic skin response and a sensor measuring heart rate variability.

In one embodiment, the first sensor, the second sensor, and the processing unit are contained in a wearable device. In one embodiment, the processing unit is separate from the first sensor and the second sensor, and the processing unit receives the first measurement signal and the second measurement signal using a wireless receiver in communication with the processing unit. In one embodiment, the processing unit and the wireless receiver are contained in a smartphone device. In one embodiment, at least one of the first sensor and the second sensor is in communication with a wireless transmitter.

In another aspect, the present invention relates to a system for hunger management. The system comprises a first sensor configured to measure a factor related to a user's metabolic state and provide a first measurement signal; a second sensor configured to measure a factor related to the availability of food for consumption by the user and provide a second measurement signal; and a processing unit configured to receive the first measurement signal and the second measurement signal, make a determination of the user's hunger, and provide feedback to the user concerning edible consumption tailored to the determination of the user's hunger.

In one embodiment, the first sensor is selected from the group consisting of a ketone sensor, a glucometer, a semi-continuous weight sensor, and an activity sensor. In one embodiment, the second sensor is selected from the group consisting of a location sensor, an artificial nose, a social data analyzer, and a daily routine analyzer. In one embodiment, the feedback is selected from the group consisting of a suggestion to eat a snack, a suggestion to refrain from eating, a message concerning the availability of unhealthy food, and an interactive distriction.

In one embodiment, the system further includes a third sensor configured to measure a factor related to the user's mood and provide a third measurement signal, and wherein the processing unit is further configured to receive the third measurement signal and incorporate the third measurement signal into the determination of the user's hunger. In one embodiment, the third sensor is selected from the group consisting of a sensor measuring galvanic skin response and a sensor measuring heart rate variability.

In one embodiment, the hunger management system is a wearable device. In one embodiment, the processing unit is separate from the first sensor and the second sensor, and the system further comprises a wireless receiver in communication with the processing unit for receiving the first measurement signal and the second measurement signal. The processing unit and the wireless receiver may be contained in a smartphone device. The system may further comprise a wireless transmitter in communication with at least one of the first sensor and the second sensor.

These and other features and advantages, which characterize the present non-limiting embodiments, will be apparent from a reading of the following detailed description and a review of the associated drawings. It is to be understood that both the foregoing general description and the following detailed description are explanatory only and are not restrictive of the non-limiting embodiments as claimed.

### BRIEF DESCRIPTION OF DRAWINGS

Non-limiting and non-exhaustive embodiments are described with reference to the following Figures in which:

FIG. 1 is a flowchart illustrating one embodiment of a method for hunger management in accord with the present invention; and

FIG. 2 is a block diagram presenting one embodiment of a system for hunger management in accord with the present invention.

In the drawings, like reference characters generally refer to corresponding parts throughout the different views. The drawings are not necessarily to scale, emphasis instead being placed on the principles and concepts of operation.

### DETAILED DESCRIPTION

Various embodiments are described more fully below with reference to the accompanying drawings, which form a part hereof, and which show specific exemplary embodiments. However, embodiments may be implemented in many different forms and should not be construed as limited to the embodiments set forth herein; rather, these embodiments are provided so that this disclosure will be thorough and complete, and will fully convey the scope of the embodiments to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one embodiment of the invention. The appearances of the phrase “in one embodiment” in various places in the specification are not necessarily all referring to the same embodiment.

Some portions of the description that follow are presented in terms of symbolic representations of operations on non-transient signals stored within a computer memory. These descriptions and representations are the means used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the present invention include process steps and instructions that could be embodied in software, firmware or hardware, and when embodied in software, could be downloaded to reside on and be operated from different platforms used by a variety of operating systems.

The present invention also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein, and any references below to specific languages are provided for disclosure of enablement and best mode of the present invention.

In addition, the language used in the specification has been principally selected for readability and instructional purposes and may not have been selected to delineate or circumscribe the inventive subject matter. Accordingly, the disclosure of the present invention is intended to be illustrative, but not limiting, of the scope of the invention, which is set forth in the claims.

### Introduction

Embodiments of the present invention coach a person to lead a healthier lifestyle based on the contextual detection and interpretation of hunger. By combining physiological and environmental signals, embodiments of the present invention make an assessment of a person's hunger state and additionally differentiate between a state of hunger triggered by bodily needs and a hunger state induced by environmental stimuli. Coaching is provided to the user depending on the assessment and source of the hunger state; typically a directive to eat a healthy snack at an appropriate time.

### Embodiments

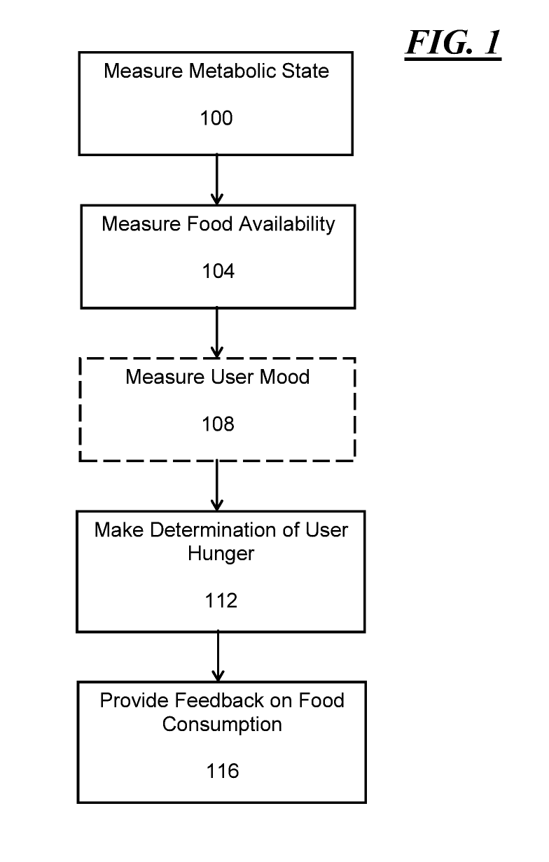

FIG. 1 is a flowchart illustrating an exemplary embodiment of a method for hunger management with a computing unit in accord with the present invention.

A factor related to a user's metabolic state is measured using at least one first sensor configured to provide a first measurement signal (Step 100). There are various factors related to a user's metabolic state that are suitable for use with the present invention, including but not limited to fat metabolism, the user's body glucose level, variations in the user's weight, and the user's energy expenditure.

The form of the first sensor will vary depending on the metabolic factor under measurement. For example, when the factor is fat metabolism, the first sensor may be an “e-nose” or another sensor for detecting ketosis. When the factor is glucose level, the first sensor may be a glucometer that measures glucose from the user's blood, tears, or sweat using chemical reactions, reflected light (e.g., a photoplethysmograph [PPG]), etc. When the factor is user weight, the first sensor may be a weight sensor placed in an article of clothing (e.g., a shoe), a piece of furniture (e.g., a desk chair), a floor, a door frame, etc. If the factor is body temperature, the first sensor may be a thermometer in contact with the skin or another sensor for measuring body temperature. When the factor is energy expenditure, the first sensor may be an accelerometer (e.g., in a carried phone or a wearable device) that indirectly measures the user's energy expenditure by measuring the user's physical activity, a cardiometer that indirectly measures the user's energy expenditure by measuring the user's heart rate, or a sensor such as a calorimeter that directly measures the user's energy expenditure.

A factor related to the availability of food for consumption by the user is measured using at least one second sensor configured to provide a second measurement signal (Step 104). There are various factors related to the availability of food for consumption by the user that are suitable for use with the present invention, including but not limited to the user's location (and associated proximity to restaurants, food stores, and other sources of food), ambient aromas (originating from nearby restaurants, food items, etc.), the user's social data feed (indicative of the user's plans to visit restaurants, planned and historic food consumption, etc.), and data concerning the user's daily routine (again, indicative of the user's plans to visit restaurants, planned and historic food consumption, etc.).

The form of the second sensor will vary depending on the factor related to the availability of food for consumption under measurement. For example, when the factor is the user's location then the second sensor may be a positioning sensor such as a GPS sensor, a GALILEO, or a GLONASS sensor. When the factor is an ambient aroma, the second sensor may be, e.g., an “e-nose” or other sensor for detecting aromas. When the factor is the user's social data feed, the second sensor may be a subscriber to one or more RSS feeds providing the user's social data, a web scraper configured to monitor one or more webpages providing the user's social data, or another social data interface. When the factor is data concerning the user's daily routine, the second sensor may be, e.g., an interface to the user's calendar or the user's historical position information.

In some embodiments, a factor related to the user's mood is measured using at least one third sensor configured to provide a third measurement signal (Step 108). There are various factors related to the user's mood that are suitable for use with the present invention, including but not limited to the variance in the user's heart rate and the user's galvanic skin response.

The form of the third sensor will vary depending on the factor related to the user's mood under measurement. For example, when the factor is the variance in the user's heart rate, then the third sensor may be, e.g., a PPG configured to measure heart rate. When the factor is the user's galvanic skin response, then the third sensor may be, e.g., a galvanic skin response sensor.

A processing unit configured to make a determination of the user's hunger receives the first and second measurement signals and, where applicable, the third measurement signal as well (Step 112). Using the received measurement signals, the processing unit makes a determination of the user's hunger. The hunger may be determined to be “intrinsic,” i.e., deriving from the body's genuine state of hunger; “extrinsic,” i.e., deriving from an outside stimulus (e.g., such as the aroma of freshly-prepared food); “lasting” (i.e., persisting for a certain time); “incipient” (i.e., recently occurring, perhaps due to a stimulus), and the intensity of the hunger may be quantified as well (“low”, “medium”, “high”, etc.).

The determination of hunger is generally dependent on the form of the first sensor and, accordingly, the form and content of the first measurement signal. When the first sensor is a glucometer, the determination of hunger is generally inversely related to the measured blood sugar level (i.e., “high” when the blood sugar level is “low”). If the first sensor is a weight sensor, the determination of hunger is generally inversely related to measurements of short term weight change (i.e., “high” when weight “decreases”). When the first sensor is an accelerometer or a cardiometer, the determination of hunger is directly related to the time integral of the sensor output. It can be assumed that each meal resets the user's hunger to a “zero” state.

Once the determination of hunger is made, feedback is provided to the user concerning edible consumption that is tailored to the determination of the user's hunger (Step 116). As noted above, the determination may be a multi-factor determination (e.g., “intense”, “incipient” and “extrinsic”), and the feedback may account for or weight all of these factors.

The feedback may take a variety of forms, depending on the current determination of the user's hunger. For example, when the intensity of the user's hunger is determined to be “high,” then the feedback may direct the user to eat a snack. If the intensity of the user's hunger is “medium,” the system may instead encourage or reward the user for refraining from snacking.

The feedback may vary depending on the “intrinsic” or “extrinsic” nature of the hunger. Generally speaking when the hunger is “low” and “extrinsic,” the feedback may take the form of distracting the user from the sensation of hunger, or motivating the user to refrain from snacking. When the hunger is “medium” and “extrinsic,” the feedback may suggest that the user have a healthy snack if the time until the user's next meal is significant.

When the hunger is “high” and “intrinsic,” then the feedback may suggest that the user eat a healthy meal. However, if the “high” hunger is “extrinsic” because it coincides with the user watching a commercial advertising junk food or “incipient” because the user is near a fast food restaurant, the feedback may be a warning cautioning the user to avoid fatty foods, a distraction such as a game or video clip, or a motivation helping the user visualize his weight loss progress or reminding the user of his recent successes at resisting cravings for unhealthy foods.

Certain embodiments may also utilize relevant extrinsic data, such as the user's schedule. Given the schedule data and current measurements from one or more of the aforementioned sensors, a future prediction of the user's hunger may be made for various events and times coinciding with the user's schedule. If, for example, it is expected that the user's hunger will be high at the end of a meeting, then the user can be advised in advance of the meeting to bring a healthy snack to the meeting or eat a high protein meal in advance of the meeting to boost satiety. In another embodiment, if the user is travelling by car, then the coaching application can advise (on request or proactively) when and where the user should stop for meals using a combination of GPS information (e.g., Google Maps route data), available lunch locations, and a model of how the user's hunger state (intrinsic, extrinsic, etc.) progresses while travelling.

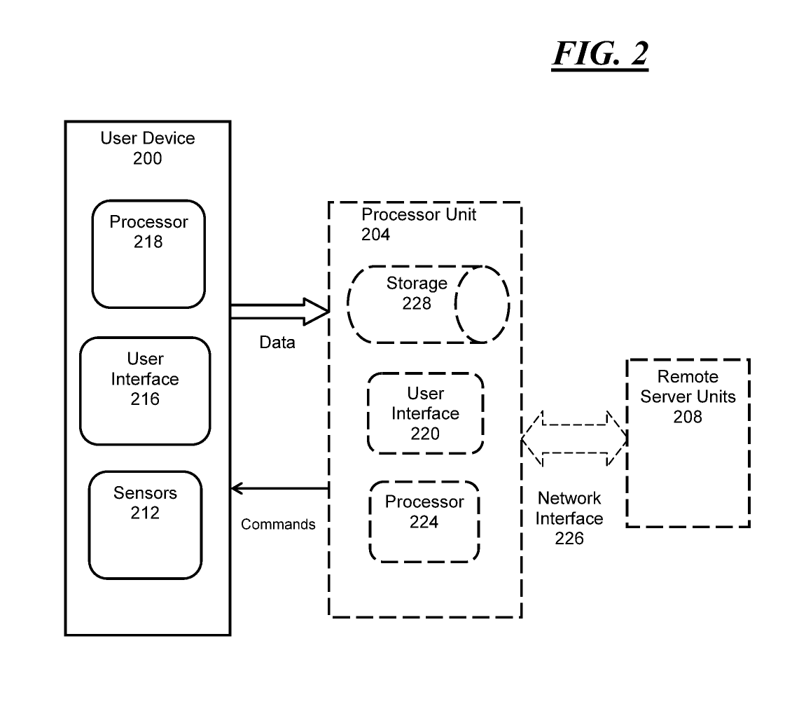

FIG. 2 is a block diagram illustrating one embodiment of a system for hunger management including a user device 200, an optional processor unit 204, and optional remote server units 208.

The user device 200 is responsible for collecting data concerning the user's activity, hunger, location, etc., and includes an interface 216 for collecting information from and/or providing feedback to the user. In some embodiments the user device 200 interoperates with a processor unit 204 that is responsible for making determinations of hunger and feedback for delivery to the user, as discussed above.

The user device 200 can take a variety of forms, such as a smartwatch, bracelet, pendant, strap, a smartphone that includes an app, or another computing resource(s). The processor unit 204 can likewise take a variety of forms, such as a server computer, desktop computer, laptop computer, tablet, phablet, smartphone, or another computing resource(s).

In some embodiments the user device 200 and the processor unit 204 are controlled by different users. For example, the user device 200 may be a smartwatch worn by a user while the processor unit 204 may be a hosted service operated by another user, such as vendor of user devices. In other embodiments, the functionality of the user device 200 and the processor unit 204 are offered by the same device. In still other embodiments, the processing capabilities of the processor unit 204 may be implemented across the processor unit 204 and one or more additional computing devices, such as optional remote server units 208. The following discussion assumes the user device 200 and the processor unit 204 to be separate physical devices for convenience, although this should not be construed to be limiting as to the overall scope of the present invention.

As illustrated, the user device 200 includes at least two sensors 212, a user interface 216, and a processor 218. As discussed above, the sensors 212 include at least a first sensor configured to measure a factor related to a user's metabolic state and provide a first measurement signal and a second sensor configured to measure a factor related to the availability of food for consumption by the user and provide a second measurement signal. The exact form of the first sensor will vary according to the metabolic state factor being measured as discussed above and may include, but is not limited to, an “e-nose,” glucometer, PPG, weight sensor, accelerometer, calorimeter, etc. Similarly, the exact form of the second sensor will vary according to the food availability factor being measured as discussed above and may include, but is not limited to, a GPS sensor, an “e-nose”, a subscriber to an RSS feed of social media information, an interface to the user's historical position information, etc. As discussed above, in some embodiments the sensors 212 also include a third sensor for measuring factors related to the user's mood, such as a PPG or a galvanic skin response sensor. Other sensors 212 may include an audio sensor, a video sensor, a respiration sensor, etc.

The user interface 216 can take many forms, but is typically appropriate to the form of the user device 200. Typical user interfaces 216 include a speech generator, a display (LCD, LED, CRT, E-Ink, etc.), a projector, a keyboard (physical or virtual), a speech recognition system, a touchscreen, etc. The processor 218 may be, e.g., an ARM-based or x86-based general purpose processing unit.

The processor 218 is configured to receive the first measurement signal and the second measurement signal, and in some embodiments the third measurement signal, and to make a determination of the user's hunger. The hunger may be determined to be “intrinsic,” i.e., deriving from the body's genuine state of hunger; “extrinsic,” i.e., deriving from an outside stimulus (e.g., such as the aroma of freshly-prepared food); “lasting” (i.e., persisting for a certain time); “incipient” (i.e., recently occurring, perhaps due to a stimulus), and the intensity of the hunger may be quantified as well (“low”, “medium”, “high”, etc.).

Once the determination of hunger is made, the processor 218 is further configured to provide feedback to the user via the user interface 216 concerning edible consumption that is tailored to the determination of the user's hunger. Again, the determination may be a multi-factor determination (e.g., “intense”, “incipient” and “extrinsic”), and the feedback may account for or weight all of these factors. Accordingly, in various embodiments coaching rules may be implemented as single factor rules resulting in an action when the factor is satisfied, or as multi-factor rules where a plurality of factors must be satisfied before the action is taken (e.g., distract when hunger is “low” and “intrinsic”).

Certain embodiments may use fuzzy logic and soft factors, resulting in a weight for each factor in the rule. In application, a processor can sample an action according to the distribution of the weights. This makes the coaching less predictable and machine-like which should improve social bonding to the coaching device.

The feedback may take a variety of forms, depending on the current determination of the user's hunger. For example, when the intensity of the user's hunger is determined to be “high,” then the feedback may direct the user to eat a snack. If the intensity of the user's hunger is “medium,” the system may instead encourage or reward the user for refraining from snacking.

However, if the “high” hunger is “extrinsic” because it coincides with the user watching a commercial advertising junk food or “incipient” because the user is near a fast food restaurant, the feedback may be a warning cautioning the user to avoid fatty foods, a distraction such as a game or video clip, or a motivation helping the user visualize his weight loss progress or reminding the user of his recent successes at resisting cravings for unhealthy foods.

The optional processor unit 204 typically includes a user interface 220, a processor 224, a network interface 226, and a storage unit 228 which acts as a repository for the computer executable instructions that execute on the processor 224. The interface 220 may, like the interface 216, take a variety of forms that is appropriate to the particular form of the processor unit 204. The processor unit 204 may be configured to receive one or more of the aforementioned measurement signals and make a determination of hunger and a determination of feedback to be delivered to the user, either working by itself or in conjunction with the user device 200.

In operation, commands are sent from the processor unit 204 to the user device 200. Data is received by the processor unit 204 from the user device 200. Data may be transmitted to and received from a remote server unit 208 by the processor unit 204 via a network interface 226 in embodiments utilizing such remote server units 208.

Certain embodiments may also involve a processor unit in communication with various relevant extrinsic data (not shown), such as the user's schedule data, GPS data, Google Maps route data, restaurant location data, hunger models, etc. Given the schedule data and current measurements from one or more of the aforementioned sensors, a future prediction of the user's hunger may be made for various events and times coinciding with the user's schedule. If, for example, it is expected that the user's hunger will be high at the end of a meeting, then the user can be advised in advance of the meeting to bring a healthy snack to the meeting or eat a high protein meal in advance of the meeting to boost satiety. In another embodiment, if the user is travelling by car, then the coaching application can advise (on request or proactively) when and where the user should stop for meals using a combination of GPS information (e.g., Google Maps route data), available lunch locations, and a model of how the user's hunger state (intrinsic, extrinsic, etc.) progresses while travelling.

Embodiments of the present invention provide a strategy that can be integrated in coaching, dietary intake monitoring, or both. These embodiments are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

The description and illustration of one or more embodiments provided in this application are not intended to limit or restrict the scope of the present disclosure as claimed in any way. The embodiments, examples, and details provided in this application are considered sufficient to convey possession and enable others to make and use the best mode of the claimed embodiments. The claimed embodiments should not be construed as being limited to any embodiment, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the general inventive concept embodied in this application that do not depart from the broader scope of the claimed embodiments.

What is claimed is:

1. A method for hunger management, the method comprising:
measuring a factor related to a user's metabolic state using a first sensor configured to provide a first measurement signal;
measuring a factor related to the availability of food for consumption by the user using a second sensor configured to provide a second measurement signal;
receiving the first measurement signal and the second measurement signal at a processing unit configured to make a determination of the user's hunger utilizing the received first measurement signal and the received second measurement signal; and
providing feedback to the user concerning edible consumption tailored to the determination of the user's hunger.

2. The method of claim 1 wherein the first sensor is selected from the group consisting of a ketone sensor, a glucometer, a semi-continuous weight sensor, and an activity sensor.

3. The method of claim 1 wherein the second sensor is selected from the group consisting of a location sensor, an artificial nose, a social data analyzer, and a daily routine analyzer.

4. The method of claim 1 further comprising:
measuring a factor related to the user's mood using a third sensor configured to provide a third measurement signal; and
receiving the third measurement signal at the processing unit,
wherein the processing unit is further configured to incorporate the third measurement signal into the determination of the user's hunger.

5. The method of claim 4 wherein the third sensor is selected from the group consisting of a sensor measuring galvanic skin response and a sensor measuring heart rate variability.

6. The method of claim 1 wherein the feedback is selected from the group consisting of a suggestion to eat a snack, a suggestion to refrain from eating, a message concerning the availability of unhealthy food, and an interactive distraction.

7. The method of claim 1 wherein the first sensor, the second sensor, and the processing unit are contained in a wearable device.

8. The method of claim 1 wherein the processing unit is separate from the first sensor and the second sensor, and the processing unit receives the first measurement signal and the second measurement signal using a wireless receiver in communication with the processing unit.

9. The method of claim 8 wherein the processing unit and the wireless receiver are contained in a smartphone device.

10. The method of claim 8 wherein at least one of the first sensor and the second sensor is in communication with a wireless transmitter.

11. A system for hunger management, the system comprising:
a first sensor configured to measure a factor related to a user's metabolic state and provide a first measurement signal;
a second sensor configured to measure a factor related to the availability of food for consumption by the user and provide a second measurement signal; and
a processing unit configured to receive the first measurement signal and the second measurement signal, make a determination of the user's hunger, and provide feedback to the user concerning edible consumption tailored to the determination of the user's hunger.

12. The system of claim 11 wherein the first sensor is selected from the group consisting of a ketone sensor, a glucometer, a semi-continuous weight sensor, and an activity sensor.

13. The system of claim 11 wherein the second sensor is selected from the group consisting of a location sensor, an artificial nose, a social data analyzer, and a daily routine analyzer.

14. The system of claim 11 further comprising a third sensor configured to measure a factor related to the user's mood and provide a third measurement signal, and wherein the processing unit is further configured to receive the third measurement signal and incorporate the third measurement signal into the determination of the user's hunger.

15. The system of claim 14 wherein the third sensor is selected from the group consisting of a sensor measuring galvanic skin response and a sensor measuring heart rate variability.

16. The system of claim 11 wherein the feedback is selected from the group consisting of a suggestion to eat a snack, a suggestion to refrain from eating, a message concerning the availability of unhealthy food, and an interactive distraction.

17. The system of claim 11 wherein the hunger management system is a wearable device.

18. The system of claim 11 wherein the processing unit is separate from the first sensor and the second sensor, and the system further comprises a wireless receiver in communication with the processing unit for receiving the first measurement signal and the second measurement signal.

19. The system of claim 18 wherein the processing unit and the wireless receiver are contained in a smartphone device.

20. The system of claim 18 wherein the system further comprises a wireless transmitter in communication with at least one of the first sensor and the second sensor.
